(19) United States
(12) Patent Application Publication
(10) Pub. No.: US 2008/0274888 A1
(43) Pub. Date: Nov. 6, 2008

(54) N-Acetylcysteine Amide (Nac Amide) for Enhancing Plant Resistance and Tolerance to Environmental Stress

(76) Inventor: Glenn A. Goldstein, New York, NY (US)

Correspondence Address: MINTZ LEVIN COHN FERRIS GLOVSKY & POPEO;ATTN: PATENT INTAKE CUSTOMER NO. 35437, ONE FINANCIAL CENTER, BOSTON, MA, 02111, US

(21) Appl. No.: 11/912,296

(22) PCT Filed: Apr. 21, 2006
(86) PCT No.: PCT/US06/15021

§ 371 Date: Nov. 8, 2007

Related U.S. Application Data
(60) Provisional application No. 60/673,560, filed on Apr. 21, 2005.

(51) Int. Cl. A01N 37/18; A01N 37/02; C07C 229/26; A01P 21/00

(52) U.S. Cl. 504/149; 504/320; 562/556

(57) ABSTRACT

The potent antioxidant N-acetylcysteine amide (NAC amide), or a physiologically acceptable derivative, salt, or ester thereof, is topically or exogenously applied to a plant, or part thereof, to reduce or prevent adverse reactions of plants and crops to environmental biotic and abiotic stresses, such as extremes of temperature, drought, humidity, frost, rain, as well as the presence or invasion of a variety of pests and pathogens. Such environmental stresses can result in oxidative stress and the correlated production (and buildup) of free radicals in plant cells, which damages plant cells and tissues and can lead to plant death. NAC amide reduces, prevents, alleviates, or otherwise counteracts such oxidative stress and free radical production, which adversely effect the overall growth and viability of the plant.

### FIELD OF THE INVENTION

The present invention generally relates to the treatment of plants or crops with an environmentally safe antioxidant to allow them to become more resistant or tolerant to a variety of environmental stresses, including, but not limited to, plant pests, pathogens, adverse weather, soil, growth and maintenance conditions.

### BACKGROUND OF THE INVENTION

Like most living organisms, plants are subjected to a variety of environmental stresses and assaults, which are both biotic and abiotic. Biotic stresses include pests such as insects, arachnids and nematodes and pathogens such as bacteria, viruses, fungi and mycoplasms. Abiotic stresses include extremes in temperature and weather conditions, such as drought, frost, excess rain, moisture and heat. Each year these stresses result in billions of dollars worth of vegetative loss resulting from damaged or reduced crop production. Thus, controlling the adverse effects of such stresses on valued plants and crops is both an economic and environmental concern.

Since World War II, control efforts to protect plants against environmental biotic and abiotic stress factors have primarily utilized synthetic toxic chemicals, e.g., pesticides. The annual usage of pesticides has increased to over 544 million kilograms. Pesticides, however, are expensive to bring to market and thus are expensive for widespread use. Moreover, because of their persistence in the environment and their potential toxicity, pesticides present a continually growing health risk to animal and humans.

The economic costs and health risks associated with the use of pesticides have led to an ever-increasing emphasis on alternative strategies to protect plants and crops. One such alternative strategy has been to search for and develop methods that allow plants to increase their own defense mechanisms. To this end, plants have been molecularly engineered to express proteins, enzymes and polypeptides that provide resistance to numerous pests, pesticides and environmental stresses.

It has been known for some time that certain stressful stimuli will increase a plant's resistance to pathogens. For example, in 1940, Muller and Borges discovered phytoalexins. (Muller, K. O. and Borges, H. Arb. Biol. Reichsanst Land-u Forstwiss. 23:189-231 (1940)). The discovery of phytoalexins provided the biochemical explanation for what had been observed to be an inducible defense response by the plant. Subsequently it has been shown that exposure to a variety of biotic or abiotic stresses (i.e., exo-elicitors) will cause a plant to synthesize and accumulate phytoalexins. These phytoalexins display antifeedant and antibiotic properties, which are protective to the plant and which, in turn, are toxic to fungi, bacteria, higher-plant cells, and also animal cells. (J. Ebel, Phytoalexin Synthesis: The Biochemical Analysis of the Inductive Process, Ann. Rev. Phytopathol, 24:235-64, 1986). These exo-elicitors may also induce other chemical defense mechanisms in addition to phytoalexins, for example, protease inhibitors and hormone mimics.

Biotic exo-elicitors that have been studied include: Phytophthora megasperma var. sojae, (a fungus), (Klarman, W. L., Netherlands Jour. Plant Pathol., 74:171-175 (1968); Chamberlain, D. W. and J. D. Paxton, Phytopathology, 58:1349-1350 (1968)); Meloidogyne incognita (a nematode), (Kaplan, D. et al., Physiol. Plant Pathol., 16:309-318, 1980); Pseudomonas syringae pv. glycinea (a bacterium) (Holliday, M. J. et al, Physiol. Plant Pathol., 18:279-287, 1981); several species of insects (Kogan, M. and J. Paxton, in: P. A. Hedin (ed.), Plant Resistance to Insects, Amer. Chem. Soc., Wash., D.C. 1983); Tetranychus urticae (a mite) (Hildebrand, D. F. et al., Jour. Econ. Entomol., 79:1459-1465, 1986); and Epilachna varivestis (an insect) (Chiang, H. S. et al., Jour. Chem. Ecol., 13:741-749, 1987).

Although such biotic exo-elicitors have been shown experimentally to increase plant resistance, they may themselves be pests or pathogens of plants. Further, large-scale production of biotic exo-elicitors that display a uniform activity in a concentration that is necessary for practical use would be difficult and costly under the best of circumstances. Thus, at present, biotic exo-elicitors do not appear to be a satisfactory alternative to toxic pesticides.

Abiotic exo-elicitors have also been identified among: fungicides and fungicidal decomposition products (Reilly, J. J. and W. L. Klarman, Phytopathology, 62:1113-1115, 1972). Maneb, ethylenediamine, polyethylene (thiocarbamoyl) monosulfide (PTM) and benomyl are representative of such fungicides. Ultraviolet irradiation was active in soybean (Bridge, M. A. and W. L. Klarman, Phytopathology, 63: 606-609, 1973). Other abiotic exo-elicitors include mercuric chloride (Moesta, P. and H. Grisebach, 286:710-711, 1980); acifluorfen and oxyfluorfen herbicides (Komives, T. and J. E. Casida, Jour. Agric. Food Chem., 31:751-755, 1983); dithiothreitol (DTT), N-ethylmaleimide (NEM), p-hydroxymercuribenzoate (PHMB) and p-chloromercuribenzenesulfonic acid (PMBS) (Stoessel, P. Planta, 160:314-319, 1984); and a glucan molecule (Grisebach, H. et al., UCLA Symp. Mol. Cell. Biol., Ser. 22: 275-290, 1985). Unfortunately, these experimental, abiotic, exo-elicitors persist in the environment and are toxic to both plant and animal living organisms. Thus, while useful for study, these compounds and substances do not avoid or diminish the problems already presented by the toxic pesticides.

For many purposes in agriculture and related endeavors it is desired to treat plants with exogenous chemical substances of various kinds. An exogenous chemical substance as defined herein is a chemical substance, whether naturally or synthetically obtained, which is applied to a plant with the intent or result of delivering the substance to one or more sites in the plant where the substance expresses some desired biological activity. Examples of exogenous chemical substances include, but are not limited to, chemical pesticides (such as herbicides, algicides, fungicides, bactericides, viricides, insecticides, miticides, nematicides and molluscicides), plant growth regulators, fertilizers and nutrients, gametocides, defoliants, desiccants, mixtures thereof and the like.

Many exogenous chemical substances or agents are applied to the foliage of a plant (i.e., the leaves and other non-woody parts of the plant that are typically above-ground), and have a site of action in the plant either close to, or remote from, the locus of application. Such substances or agents are referred to as foliar-applied exogenous chemical substances. Preferably an exogenous, foliar-applied substance or agent will efficiently and effectively be applied or delivered so as to reach the sites of action in the plant where the biological effect of the exogenous substance or agent can be utilized and functionally effective in the plant. Ideally, also, the substances or agents will be applied in a reduced rate of time without sacrificing consistency of biological effectiveness. Pressures felt by the agricultural industry to reduce pesticide, particularly herbicide, usage are well evidenced by symposia on the subject, for example, Symposium of the Weed Science Society of America, 1993, as documented in Weed Technology, 8:331-386 (1994). Reduced use rates bring rewards not only environmentally but also economically, as the cost per unit area treated decreases.

Herbicidal compositions have been described containing chemical synergists, which have been hypothesized to enhance herbicidal effectiveness by affecting the metabolic processes of a plant. Such chemical synergists include 6-benzylaminopurine, gibberellic acids, and 2-choroethylphosphonic acid, all known to have plant growth regulating activity in their own right. For example, it has been reported that if gibberellic acids are applied to growing plants at some time prior to the application of a glyphosate herbicide composition, the herbicidal effectiveness of the glyphosate is increased. However, the use of some synergists such as 6-benzylaminopurine, gibberellic acids, and 2-choroethylphosphonic acid is limited because of the need to apply the synergist days or even weeks before the application of the herbicide. Other synergists, while capable of being applied simultaneously with the herbicide, are effective only at high concentrations, e.g., 1:1 or 2:1 ratios by weight of the exogenous chemical substance to synergist.

A widely practiced method of enhancing reliability of biological effectiveness of a foliar-applied composition of an exogenous chemical substances, particularly a herbicide, is to add an enhancing agent comprising an ammonium salt, e.g., ammonium sulfate, to the composition being applied. It is well known to those practicing this method that enhanced biological effectiveness is not assured with every use; however the low cost of the method means that even if biological effectiveness is enhanced in only a small proportion, for example 1 in 5, of times the method is used, it is still worthwhile.

To provide a substance topically or to the foliage of a plant or crop in combination with an enhancing agent, it is preferred that both the substance and the enhancing agent be employed at a low use rate, while at the same time allowing the reliability of effectiveness of the topical or foliar applied exogenous substance. The biological effectiveness of an exogenous chemical substance depends upon delivery of the substance into living cells or tissues of the plant. Accordingly, the use of a low-rate enhancing agent that stimulates various biological processes in plants. (See, e.g., U.S. Pat. No. 4,436,547 to Sampson). This patent discloses that additives can be used to improve the action of agricultural chemicals. Such additives can include a carbohydrate source or organic acid to supply metabolizable energy or as precursors of amino acids and nucleotides; a vitamin or coenzyme to stimulate metabolic processes; a nucleic acid precursor to stimulate nucleic acid synthesis; a fatty acid (or fat or oil that can be degraded thereto) as precursor of molecules required in growth processes; an amino acid as structural unit for protein synthesis; and a naturally occurring plant growth regulator to affect metabolism in such a way as to render an applied pesticide or other substance more effective. In the case of herbicides, the patent discloses that “by stimulating growth and uptake of applied chemicals it is possible to enhance the activity of a number of herbicides, especially against older more established weeds.” Other agents that may enhance the biological effectiveness of a foliar-applied exogenous chemical substance involving the use of an anthraquinone compound as enhancing agent. (U.S. Pat. No. 6,172,004 to R. J. Brinker et al.).

In plants, oxidative stress is induced by a wide variety of environmental factors, including ultraviolet radiation stress, pathogen invasion (hypersensitive invasion), herbicide action, oxygen and nutrient shortage. Oxygen deprivation in plant cells results in three physiologically different states: transient hypoxia, anoxia and reoxygenation. (O. Blokhina et al., 2003, Ann. Bot. (London), 91:179-194). Reactive oxygen species (ROS), i.e., hydrogen peroxide and superoxide, are generated as a consequence of hypoxia and reoxygenation. Lipids (peroxidation of unsaturated fatty acids in membranes), proteins (denaturation), carbohydrates and nucleic acids are the main cellular components that are susceptible to damage by free radicals. The consequences of hypoxia-induced oxidative stress depend upon tissue and/or species tolerance to anoxia; upon membrane properties; upon endogenous antioxidant content; and upon the ability to induce the response in the antioxidant system. The antioxidant system in plants involves low molecular mass antioxidants, such as ascorbic acid, glutathione (GSH) and tocopherols; enzymes regenerating the reduced forms of antioxidants and ROS-interacting enzymes, such as SOD, peroxidases and catalases. Antioxidants behave as a cooperative network, in which a series of redox reactions and interactions between ascorbic acid and GSH, and ascorbic acid and phenolic compounds are known. (O. Blokhina et al., 2003, Ann. Bot. (London), 91:179-194). However, it is also known that under oxygen deprivation stress, antioxidants within the plant system are not always competent or effective in enhancing antioxidant defenses in plant cells. Thus, a plant's natural antioxidant status may not be sufficient to scavenge ROS compounds, and to protect the plant from oxygen deprivation and other environmental stresses.

Needed in the art are new compounds and methods for safely and economically treating plants, with or without the above-described enhancing agents, to protect them against adverse environmental stresses and agents that can typically adversely affect the plant's own antioxidant system. Such compounds and methods should optimally be safe themselves, not linger for too long in the environment following application and be easily and/or readily applied to plants and crops, both on a small and a larger, agriculturally convenient scale.

### SUMMARY OF THE INVENTION

The present invention provides the use of the antioxidant N-acetylcysteine amide (NAC amide), or a physiologically acceptable derivative, salt, or ester thereof, topically or exogenously applied to a plant, or part thereof, to reduce or prevent adverse reactions of plants and crops to environmental biotic and abiotic stresses, such as extremes of temperature, drought, humidity, frost, rain, as well as the presence or invasion of a variety of pests and pathogens. Such environmental stresses can result in oxidative stress and the correlated production (and buildup) of free radicals in plant cells. NAC amide reduces, prevents, alleviates, or otherwise counteracts such oxidative stress and free radical production, which cause damage to the overall growth and viability of the plant.

NAC amide, for use herein, is a biodegradable, non-pesticidal, non-toxic and environmentally compatible antioxidant. The use of NAC amide avoids problems that typically exist with use of toxic pesticides, while at the same time achieving significant practical control of environmental stresses, pests and pathogens. The application to the surface of a plant or crop of an effective amount of NAC amide as an environmentally safe antioxidant elicits a systemic and protective response in the plant or crop, which is akin to boosting, bolstering, enhancing, or augmenting the physiological defense mechanisms of the plant or crop. Thus, treatment of one portion of a plant or crop elicits a defensive or protective response throughout the plant. The effectiveness of NAC amide to elicit a defensive response can be facilitated by administering water soluble NAC amide alone, or in conjunction with one or more enhancing agents, or other antioxidant compounds, as described herein, and/or dispersed in a non-reactant, membrane permeable, carrier.

One aspect of the present invention provides a method for protecting plants and crops from environmental extremes by administering to the plants and crops a composition comprising NAC amide antioxidant. NAC amide is water-soluble and is administered exogenously, for example, by spraying, or is otherwise topically applied. For the purposes of the present invention, a composition or preparation of NAC amide can be applied by utilizing known or newly developed equipment, devices and machinery designed to treat plants and crops with exogenous agents. Aerial application is also encompassed.

Another aspect of the present invention provides the use of environmentally safe and effective compositions comprising NAC amide for the treatment of plants to control plant pests and pathogens. In a related aspect, the present invention provides a method for plant pest and pathogen control, which utilizes compositions comprising NAC amide for the treatment of plants, in which the methods are easy and economical to use and manufacture.

A further aspect of the present invention provides a water soluble composition or preparation comprising NAC amide for spraying or topically applying to plants and plant foliage, which is absorbed into the plant and protects and/or tolerizes the plant from one or more of excesses of heat (drought), moisture, precipitation (flooding, snow), frost, hail, salinity, minerals, pests and pathogens.

Another aspect of the invention provides a process for treating a plant with exogenous antioxidant, NAC amide, or a derivative, salt or ester thereof, alone or in combination with one or more enhancing agents, comprising the steps of (a) applying to surfaces or foliage of the plant a composition comprising NAC amide, or a derivative, salt, or ester thereof; and (b) applying a biologically effective amount of the NAC amide-containing composition to the same surfaces or foliage. When an enhancing agent is used, such agent is employed in a substantially non-phytotoxic amount, e.g., at least about 0.25 g/ha, which does not substantially antagonize or depress the biological effectiveness of the NAC amide.

In another of its aspects, the present invention provides a method for increasing the resistance or tolerance of a plant to a biotic or abiotic environmental stress comprising the step of administering to the surface of said plant N-acetylcysteine amide (NAC amide) in an amount effective to induce said resistance or tolerance in the plant.

In another aspect, the present invention provides a method for increasing the resistance or tolerance of a plant to one or more of pests, pathogens, or abiotic environmental stresses, comprising the step of spraying the above ground surface of the plant with a solution containing N-acetylcysteine amide (NAC amide) in an amount effective to increase the plant's resistance or tolerance.

In yet another aspect, the invention provides a method for increasing the resistance or tolerance of a plant to one or more of pests, pathogens, or abiotic environmental stresses, comprising the steps of applying N-acetylcysteine amide (NAC amide) to the stem of a plant in an amount sufficient to increase the plant's resistance or tolerance.

In a further aspect, the present invention provides a process for treating a plant or crop with exogenous N-acetylcysteine amide (NAC amide) or a derivative, salt or ester thereof, alone or in combination with one or more enhancing agents, comprising the steps of (a) applying to surfaces or foliage of the plant or crop a composition comprising NAC amide, or a derivative, salt or ester thereof; and (b) applying a biologically effective amount of the NAC amide-containing composition to the same plant or crop surfaces or foliage. In a related aspect, the NAC amide is water-soluble. In a further related aspect, the composition of step (a) comprises an enhancing agent in a substantially non-phytotoxic amount that does not substantially antagonize or depress the biological effectiveness of the NAC amide.

In another aspect, the present invention provides methods and compositions in which NAC amide supplements GSH that is produced by plants that have been subjected or exposed to environmental and physiological stresses. In accordance with this aspect, the provision of NAC amide can allow the plant to withstand stresses that it has not previously been capable of withstanding. In a related aspect, providing NAC amide allows transgenic plants to survive and thrive in natural environments.

In another aspect, the present invention provides a method of supplying NAC amide to plants that are or have been deprived of oxygen. In this aspect, the production or buildup of reactive oxygen species can be prevented or counteracted.

In another aspect, the present invention provides NAC amide to interact with salicylic acid-binding protein 2 (SABP2), which together can boost, bolster, enhance, or augment the natural defense and immune systems of plants to protect and defend the plants against environmental, physiological and oxidative stresses and insults. In a related aspect, NAC amide may serve as a plant hormone or intracellular or extracellular messenger to protect, boost, bolster, enhance, or augment the natural defense and immune systems of plants to protect and defend the plants against a variety environmental, physiological and oxidative stresses and insults as described herein.

In another of its aspects, the present invention provides a method involving NAC amide to increase the content or amount of antioxidants or phytochemicals in one or more of seeds, fruits, plants, or other plant-related products, for example, pulp for paper. In accordance with this aspect, the method will result in plants, seeds, fruits, or plant products that are healthier for animal and human consumption, as well as more economically and commercially useful, e.g., higher yields of ethanol or higher yields of harvestable or useful foodstuffs per acre, etc.

Additional aspects, features and advantages afforded by the present invention will be apparent from the detailed description and exemplification hereinbelow.

### DETAILED DESCRIPTION OF THE INVENTION

The present invention involves the use of an effective, environmentally safe antioxidant, N-acetylcysteine amide (NAC amide), alone or in combination with another agent, to permit plants to become more resistant to, or tolerant of, environmental stresses.

Glutathione N-acetylcysteine amide (NAC amide), the amide form of N-acetylcysteine (NAC), is a novel low molecular weight thiol antioxidant and a Cu2+ chelator. NAC amide provides protective effects against cell damage in its role as a scavenger of free radicals. In mammalian red blood cells (RBCs), NAC amide has been shown to inhibit tert-butylhydroxyperoxide (BuOOH)-induced intracellular oxidation and to retard BuOOH-induced thiol depletion and hemoglobin oxidation in the RBCs. This restoration of thiol-depleted RBCs by externally applied NAC amide was significantly greater than that found using NAC. Unlike NAC, NAC amide protected hemoglobin from oxidation. (L. Grinberg et al., Free Radic Biol Med., 2005 Jan. 1, 38(1):136-45). In a cell-free system, NAC amide was shown to react with oxidized glutathione (GSSG) to generate reduced glutathione (GSH). NAC amide readily permeates cell membranes, replenishes intracellular GSH, and, by incorporating into the cell's redox machinery, protects the cell from oxidation. Because of its neutral carboxyl group, NAC amide possesses enhanced properties of lipophilicity and cell permeability. (See, e.g., U.S. Pat. No. 5,874,468 to D. Atlas et al.). NAC amide is also superior to NAC and GSH in crossing the cell membrane, as well as the blood-brain barrier.

NAC amide may function directly or indirectly in many important biological phenomena, including the synthesis of proteins and DNA, transport, enzyme activity, metabolism, and protection of cells from free-radical mediated damage. NAC amide is a potent cellular antioxidant responsible for maintaining the proper oxidation state within cells. NAC amide is synthesized by most cells and can recycle oxidized biomolecules back to their active reduced forms. As an antioxidant, NAC amide may be as effective, if not more effective, than GSH.

In accordance with the present invention, stresses to plants are intended to include oxidative damage and oxygen deprivation stresses in which the antioxidant system of plants is called upon to combat and overcome. The present invention embraces methods and compositions in which NAC amide is provided as an antioxidant to replace or supplement GSH production to remove reactive oxygen species and to protect plant cells from the effects of oxidative stress on cellular lipids, proteins, enzymes, carbohydrates and nucleic acids.

In one embodiment of the present invention a method is provided for increasing the resistance of plants to pests or pathogens by administering to the surface of the plant an environmentally safe and effective amount of the antioxidant NAC amide, or derivatives thereof. The administration of NAC amide to the surface of plants induces in the plant or crop a systemic, protective response. Application to the surface of the plant may involve leaves, stems, roots, flowers, buds, stalks, foliage, etc. In addition, NAC amide can be applied in a variety of ways, including, but not limited to, drenching the root system of the plant, spraying the plant with a solution, composition, or preparation containing an effective amount of water-soluble NAC amide, or direct application to the stem, leaves, stalk, etc. of the plant with NAC amide contained in a suitable carrier. It is to be understood that the term plant is intended to encompass crops of various types and varieties.

In another embodiment, the present invention encompasses a method for protecting plants and crops from environmental extremes by administering to the plants and crops a composition or preparation comprising water soluble NAC amide antioxidant. NAC amide is administered or applied exogenously, for example, by spraying, or other type of topical application. For the purposes of the present invention, a composition or preparation of NAC amide can be applied by utilizing known or newly developed equipment, devices and machinery designed to treat plants and crops with exogenous agents. Aerial application is encompassed, as are brushing and dusting plant surfaces with an NAC amide-containing composition or preparation.

In another embodiment, the present invention encompasses the use of environmentally safe and effective compositions comprising NAC amide for the treatment of plants to control plant pests and pathogens. In a related embodiment, the present invention provides a method for plant pest and pathogen control which utilizes compositions and preparations comprising NAC amide for the treatment of plants and crops, in which the methods are easy and economical to use and manufacture.

In another embodiment, the present invention embraces a water soluble composition or preparation comprising NAC amide for spraying or topically applying to plants and plant foliage and surfaces, which is absorbed into the plant and protects and/or tolerizes the plant from one or more of excesses of heat (drought), moisture, precipitation (flooding, snow), frost, hail, salinity, minerals, pests and pathogens. In this embodiment, the invention involves a process for treating a plant with exogenous antioxidant, NAC amide, or a derivative thereof, alone or in combination with one or more enhancing agents, comprising the steps of (a) applying to surfaces or foliage of the plant a composition comprising NAC amide, or a derivative thereof; and (b) applying a biologically effective amount of the NAC amide-containing composition to the same plant surfaces or foliage. An enhancing agent can also be used. In such cases, the enhancing agent is employed in a substantially non-phytotoxic amount, e.g., at least about 0.25 g/ha, which does not substantially antagonize or depress the biological effectiveness of the NAC amide. An enhancing agent can general enhance the reliability of effectiveness of exogenously, foliar-applied NAC amide and at a low use rate.

In one embodiment, the enhancing agent is an anthraquinone compound, which is defined as a six-membered carbon ring having double bonded oxygen atoms attached to two carbon atoms in that ring, and two phenyl rings fused to the six-membered carbon ring, optionally containing one or more substitutions on one or more of the rings. The oxygen atoms can be in the para-configuration, i.e. attached directly opposite each other on the six-membered carbon ring. Suitable classes and types of anthraquinone compounds are described in U.S. Pat. No. 6,172,004 to R. J. Brinker et al. By “substantially non-phytotoxic”, in the case of an anthraquinone compound as enhancing agent, is meant that an anthraquinone compound, if it were to be applied in the absence of NAC amide, causes no significant injury, growth reduction, herbicidal effect or readily visible symptoms to the plant.

If, in the methods of the present invention, an enhancing agent is used along with application of a composition or preparation comprising NAC amide as antioxidant, the application of the NAC amide composition or preparation and the application of the enhancing agent, e.g., an anthraquinone compound, can occur either sequentially or simultaneously. In the case of simultaneous application, the NAC amide containing composition or preparation and enhancing agent can be components of a single composition adapted for such application.

In another embodiment of the present invention, a plant treatment composition is provided comprising NAC amide antioxidant, such that, when the composition is applied to foliage of a plant, with or without prior dilution, dispersion or dissolution in an application medium, the exogenously applied NAC amide is in a biologically effective amount to protect or tolerize the plant to one or more biotic and abiotic environmental stresses. Typically the application medium appropriate for compositions of the invention is water. Environmental stresses include biotic and abiotic stresses. Biotic environmental stresses embrace, without limitation, pests such as insects, arachnids and nematodes and pathogens such as bacteria, viruses, fungi and mycoplasms. Abiotic environmental stresses include, without limitation, extremes in temperature and weather conditions, such as drought, frost, excess rain, moisture and heat, and stressful physiological conditions, such as excess salinity, minerals, poor nutrients in soil or growth medium, and the like.

The use of an enhancing agent, such as anthraquinone, in conjunction with the application of NAC amide in the present invention has several benefits and advantages. One benefit is that the invention provides a process for treating plants with a foliar-applied exogenous NAC amide composition that enhances the reliability of effectiveness of the NAC amide composition. In addition, the reliability of the effectiveness of foliar-applied NAC amide is enhanced in plants at very low use rates, for example, from about 0.25 to about 250 g/ha, so that, among other advantages, it becomes economically feasible to include the agent in a concentrate composition without excessively reducing the loading therein of the NAC amide itself. A further benefit of the invention is provision of a composition containing NAC amide antioxidant that, through selection of an appropriate anthraquinone compound for inclusion in such composition, is better adapted for specific uses than are currently employed.

In another embodiment, the present invention encompasses a method and composition in which NAC amide supplements GSH that is produced by plants that have been subjected or exposed to one or more environmental or physiological stresses, including oxidative stress. The provision of NAC amide can allow the plant to withstand stresses that it has not previously been capable of withstanding. In accordance with this method, NAC amide can supplement the antioxidant substances naturally made in the stressed plants, or it can provide the necessary antioxidant component to the antioxidant system of a plant, when the plant's natural antioxidant system is defective, injured, harmed, or is otherwise incapable of full or adequate function.

The present invention includes NAC amide provided to naturally growing plants, or parts thereof, seeds, fruits, cuttings, (i.e., plant materials) and to transgenic plants, including parts thereof, seeds, fruits, cuttings, (i.e., transgenic plant materials) to allow these plants and plant materials to survive and thrive in natural and manmade environments.

In another embodiment, the present invention encompasses a method of supplying NAC amide to plants that are or have been deprived of oxygen due to environmental stresses, increased salinity, or other adverse conditions, so that the production or buildup of reactive oxygen species is prevented or counteracted. Supplying NAC amide to plants is particularly useful when the plant's own antioxidant system, e.g., the production of GSH, is prevented from or is incapable of functioning. In accordance with this embodiment, NAC amide supplements the stimulation of GSH production in plants that have been exposed to stresses and can allow the NAC amide-treated plants to withstand stresses that they would not normally withstand, for example, due to a limited amount of GSH antioxidant. This embodiment is particularly applicable for transgenic plants. Supplying NAC amide to transgenic plants can allow such plants to survive and thrive, since in many instances they have been found to be unable to generate GSH internally following environmental insults and stresses, including oxidative stresses.

In another embodiment, the present invention encompasses a method in which NAC amide interacts with salicylic acid-binding protein 2 (SABP2). Both NAC amide and SABP2 together can boost, bolster, enhance, or augment the natural defense and immune systems of plants to protect and defend the plants against environmental, physiological and oxidative stresses and insults. In addition, NAC amide may serve as a plant hormone or intracellular or extracellular messenger to protect, boost, bolster, enhance, stimulate, or augment the natural defense and immune systems of plants to protect and defend the plants against a variety environmental, physiological and oxidative stresses and insults.

In another embodiment, the present invention embraces a method of adding NAC amide to plants to increase the content or amount of antioxidants or phytochemicals in one or more of seeds, fruits, whole plants, plant parts, or products derived or made from the plants. For example, treating a plant with NAC amide allows the plant itself, or seed, fruit, or plant part, or a product derived or obtained from the plant, to be healthier for animal and human consumption, as well as more economically and commercially useful, e.g., higher yields of ethanol or higher yields of harvestable or useful foodstuffs per acre, etc. For example, the pulp for paper could be made more commercially useful by the practice of the present invention.

It is to be understood that the methods and compositions of the invention comprising NAC amide may be utilized or produced in combination with other materials and compounds that may enhance the utility of the method, e.g., fertilizers, other antioxidants, other chemicals or additives for plant and crop growth and/or production. Of particular advantage are those additive or adjunct materials and compounds and the like that are non-toxic and safe for the environment and animal, including human, consumption and exposure.

Compositions comprising NAC amide according to this invention can take the form of dilute ready-to-apply solutions or dispersions, referred to herein as spray compositions, as well as liquid and solid concentrates which, on dilution, dispersion or dissolution in water or other carrier, provide such spray compositions. In making a liquid or solid concentrate, NAC amide is typically blended by the manufacturer with suitable formulation ingredients. Such ingredients are well known to those of skill in the art and their selection depends on a particular use. Illustrative further ingredients include, without limitation, solvents, surfactants, dispersants, thickening agents, antifoams, dyes, antifreezes, preservatives and the like. In an embodiment of the present invention, the process provided includes a step of adding an enhancing compound or agent during the making of a concentrate composition of NAC amide. This concentrate composition is later diluted, dissolved or dispersed in water to make a spray composition, which is then applied by spraying onto the foliage or surfaces of plants, thus providing NAC amide to protect and increase the tolerance of the plants to environmental stresses.

If, in addition to NAC amide, the enhancing agent or compound, e.g., an anthraquinone compound, is readily soluble in water, a liquid concentrate can be provided as a simple aqueous solution. If, on the other hand, the enhancing agent or compound is not readily soluble in water, various ways are known in the art of formulating liquid concentrates, including emulsifiable concentrates, suspension concentrates and aqueous emulsions, that contain NAC amide and the enhancing agent in admixture.

Of particular interest are situations in which NAC amide is water-soluble and the enhancing agent or compound is oil-soluble. In such situations, an emulsion form of the concentrate composition is formed having an aqueous phase and an oil phase, wherein NAC amide is present primarily in the aqueous phase and the enhancing agent or compound is present primarily in the oil phase, and wherein the emulsion is stabilized by means of one or more emulsifiers. The oil phase can further comprise any of a large number of organic oils and solvents known in the agricultural chemical formulation art, including paraffinic and aromatic oils, or fatty acid alkylesters such as butyl stearate, isopropyl myristate or methyl oleate. Alternatively, the oil phase can contain the enhancing agent or compound itself. Emulsion compositions of the invention include oil-in-water macroemulsions and microemulsions, water-in-oil or invert emulsions, and water-in-oil-in-water multiple emulsions.

Without wishing to be bound by theory, this invention can provide useful benefits when NAC amide relies, at least in part for its biological effectiveness, on systemic movement in plants. Systemic movement in plants can take place via apoplastic (non-living) pathways, including within xylem vessels and in intercellular spaces and cell walls, via symplastic (living) pathways, including within phloem elements and other tissues composed of cells connected sympastically by plasmodesmata, or via both apoplastic and symplastic pathways. For foliar-applied systemic NAC amide, the most important pathway is the phloem, and the present invention can provide benefits for NAC amide that is phloem-mobile.

Water-soluble NAC amide can exist in the form of a salt comprising a biologically active ion and a counterion that is biologically inert or relatively inactive. Such a salt can have a molecular weight below about 300, excluding any counterions. In certain instances, NAC amide may be applied to plants or crops in conjunction with another exogenous chemical substance or salt thereof. Especially suitable among low molecular weight salts of other exogenous chemical substances are herbicides, plant growth regulators and nematicides, in particular those having an amine, a carboxylic acid, a phosphonate or a phosphinate functional group in the biologically active ion. Among the most preferred of such salts are those having an amine group, a carboxylic acid group, and either a phosphonate or phosphinate group in the biologically active ion, including salts of glyphosate and salts of glufosinate.

Nonlimiting examples of herbicides that can be used in the method of the invention include aminotriazole, asulam, bentazon, bialaphos, bipyridyls such as paraquat, bromacil, clopyralid, cyclohexenones such as sethoxydim, dicamba, diphenylethers such as acifluorfen, fomesafen and oxyfluorfen, fosamine, glufosinate, glyphosate, hydroxybenzonitriles such as bromoxynil, imidazolinones such as imazethapyr, isoxaben, phenoxies such as 2,4-D, phenoxypropionates such as quizalofop, picloram, substituted ureas such as fluometuron, sulfonylureas such as chlorimuron, chlorsulfaron, halosulfuron and sulfometuron, and triazines such as atrazine and metribuzin. Phloem-mobile herbicides for use by the method of the invention include but are not limited to aminotriazole, asulam, bialaphos, clopyralid, cyclohexenones, dicamba, glufosinate, glyphosate, imidazolinones, phenoxies, phenoxypropionates, picloram and sulfonylureas.

Herbicidally active derivatives of the above herbicides and of other herbicides are also within the scope of the invention if applied by the method herein described. A herbicidally active derivative is any compound which is a minor structural modification, most commonly but not restrictively a salt or ester, of a herbicide, in which the compound retains the essential activity of the parent herbicide although not necessarily having a potency equal to that of the parent herbicide. Usually but not always, the derivative converts to the parent herbicide before or after it enters the treated plant, and is analogous to a pro-drug that converts to an active drug in vivo. Mixtures or co-formulations of NAC amide with one or more herbicide or an herbicidally active derivative, or with other ingredients are also within the scope contemplated by the present invention.

Although NAC amide provides excellent benefits in the form of protecting and tolerizing a plant to numerous environmental stresses, the method of the present invention can include one or more other classes of environmentally safe antioxidants, e.g., ascorbates, tocopherols, reduced glutathione and its derivatives, and cysteines (half cystines), along with NAC amide, if necessary or desired. Ascorbates may include all forms, isomers and derivatives of ascorbic acid (including Vitamin “C” or L-ascorbic acid) that have antioxidant and reducing activities or functions. Tocopherols include Vitamin “E” (2,5,7,8-tetramethyl-2-(4′,8′,12′-trimethyltridocyl)-6-chromanol), all isomers of tocopherol which have antioxidant or reducing activity and all tocopherol esters and other derivatives that have antioxidant or reducing activities or functions.

Liquid and dry concentrate formulations of the invention can optionally contain, in addition to an NAC amide-containing composition or preparation, with or without an enhancing agent or compound, other desired or useful ingredients. Other useful ingredients may include surfactants, which assist in retention of aqueous spray solutions on the relatively hydrophobic surfaces of plant leaves, as well as helping the compositions and formulations to penetrate the waxy outer layer (cuticle) of the leaf or other plant part and thus to contact living tissues within the leaf or other plant part. Surfactants can perform other useful functions as well, including serving as emulsifiers to permit one or more components of the applied compositions to be incorporated in a stable homogeneous formulation

A variety of different types or chemical classes of surfactants can be used in the compositions of this invention. Nonionic, anionic, cationic and amphoteric types, or combinations of more than one of these types, are all useful in particular situations. Among nonionic surfactants, illustrative classes include polyoxyalkylene alkyl and alkylaryl ethers, such as ethoxylated primary or secondary alcohols or alkylphenols, polyoxyalkylene alkyl esters, such as ethoxylated fatty acids, polyoxyalkylene sorbitan alkyl esters, glyceryl alkyl esters, sucrose esters, alkyl polyglycosides, and the like. Among cationic surfactants, illustrative classes include polyoxyalkylene tertiary alkylamines, such as ethoxylated fatty amines, quaternary ammonium surfactants, polyoxyalkylene alkyletheramines, and the like. (See, e.g., the polyoxyalkylene alkyletheramines as disclosed in PCT Publication No. WO 96/32839). Among amphoteric surfactants, illustrative preferred classes include polyoxyalkylene alkylamine oxides, alkylbetaines, alkyl-substituted amino acids and the like.

Hydrophobic moieties of surfactants useful in the compositions of the invention can be essentially hydrocarbon based, or can contain silicon atoms, for example, in the form of siloxane groups, or fluorine atoms, for example, as partially fluorinated alkyl or perfluoroalkyl groups. Hydrocarbon chains of surfactants useful herein typically have from about 8 to about 20, or from about 12 to about 18 carbon atoms, and are branched or unbranched, saturated or unsaturated. Polyoxyalkylene moieties of surfactants useful in compositions of the invention are preferably polyoxyethylene or polyoxyethylene-polyoxypropylene chains. Standard reference sources from which one of skill in the art can select suitable surfactants, without limitation to the above mentioned classes, include Handbook of Industrial Surfactants, Second Edition (1997) published by Gower, McCutcheon's Emulsifiers and Detergents, North American and International Editions (1997) published by MC Publishing Company, and International Cosmetic Ingredient Dictionary, Sixth Edition (1995), (or Current Edition), Volumes 1 and 2, published by the Cosmetic, Toiletry and Fragrance Association.

Other optional components of compositions of the invention include agents or components that modify color, viscosity, gelling properties, freezing point, hygroscopicity, caking behavior, dissolution rate, dispersibility, or other characteristics of the applied compositions and formulations.

As an alternative to providing an enhancing compound as a component of the NAC amide-containing formulation, the former compound can be provided in a separate composition. In such a case the composition comprising the enhancing compound is typically tank-mixed with the NAC amide-containing composition. A tank-mixed composition is prepared by the user as a single spray composition by dilution, dissolution or dispersion in water of two concentrate compositions, one containing the NAC amide-containing composition and the other containing the enhancing compound, for example anthraquinone. The two concentrate compositions can be supplied independently or in a twin-pack or other form of combined packaging. As a particular embodiment of the invention a concentrate composition is encompassed which comprises an NAC amide-containing composition, with or without an enhancing compound together with one or more surfactants. Such a composition is useful as an adjuvant for tank-mixing with other substances, as necessary or desired, prior to application to plant foliage.

Alternatively, an enhancing compound, e.g., anthraquinone, or a composition thereof, can illustratively be used as a pre-treatment or post-treatment before or after foliar application of any commercial formulation of NAC amide. When an enhancing compound is applied to foliage as a pre-treatment or post-treatment, the interval between this treatment and application of the NAC amide-containing composition should be such as to allow the enhancing compound to enhance reliability of effectiveness of the NAC amide in the composition. Such an interval is termed an “effective time period”. What constitutes an effective time period varies depending on the species of plant, or on the particular enhancing compound, among other factors. As an illustrative, non-limiting example, an interval of from 0 to about 96 hours can be an effective time period, or an interval of from 0 to about 24 hours. Where sequential application is employed, a preferred sequence is for the enhancing compound to be applied before the NAC amide-containing composition. An optimum interval can readily be determined for any combination of NAC amide, enhancing compound and plant species by preliminary testing routinely carried out in the field.

The selection of application rates for an NAC amide-containing composition, preparation, or formulation that are biologically effective is also within the skill of the ordinary agricultural practitioner. One of skill in the art will appreciate that individual plant conditions as well as weather and growing conditions, can affect the results achieved in practicing the method of the present invention. The skilled practitioner can select NAC amide application rates that are effective on a particular species at particular growth stages to achieve protection or tolerance of a given environmental stress under particular environmental conditions.

The method of the present invention in which NAC amide, more particularly a composition or preparation containing water-soluble NAC amide, or a water-soluble salt thereof, is applicable to any and all plant species on which NAC amide is biologically effective as an antioxidant or plant growth regulator. This encompasses a very wide variety of plant species worldwide. Likewise, compositions of the invention containing NAC amide can be applied to any and all plant species on which NAC amide is biologically effective. For example, annual broadleaf species on which the method and compositions of the invention can be employed include, without limitation, Abutilon theophrasti (velvetleaf), Amaranthus spp. (pigweed), Borreria spp. (buttonweed), Brassica spp. (oilseed rape, canola, indian mustard, etc.), Commelina spp. (commelina), Erodium spp. (filaree), Helianthus spp. (sunflower), Ipomoea spp. (morning glory), Kochia scoparia (kochia), Malva spp. (mallow), Polygonum spp. (wild buckwheat, smartweed, etc.), Portulaca spp. (purslane), Salsola spp. (russian thistle), Sida spp. (sida), Sinapis arvensis (wild mustard), and Xanthium spp. (cocklebur). Further and without limitation, annual narrowleaf species on which the method and compositions of the invention can be employed include, without limitation, Avena fatua (wild oat), Axonopus spp. (carpetgrass), Bromus tectorum (downy brome), Digitaria spp. (crabgrass), Echinochloa crus-galli (barnyard grass), Eleusine indica (goosegrass), Lolium multiflorum (annual ryegrass), Oryza sativa (rice), Ottochioa nodosa (ottochloa), Paspalum notatum (bahiagrass), Phalaris spp. (canarygrass), Setaria spp. (foxtail), Triticum aestivum (wheat) and Zea mays (corn or maize).

Perennial broadleaf species on which the method and compositions of the invention can be employed include, without limitation, Artemisia spp. (mugwort), Asclepias spp. (milkweed), Cirsium arvense (canada thistle), Convolvulus arvensis (field bindweed) and Pueraria spp. (kudzu). Perennial narrowleaf species on which the method and compositions of the invention can be employed include, without limitation, Brachiaria spp. (brachiaria), Cynodon dactylon (bermudagrass), Cyperus esculentus (yellow nutsedge), Cyperus rotundus (purple nutsedge), Elymus repens (quackgrass or couch), Inperata cylindrica (cogongrass or lalang), Lolium perenne (perennial ryegrass), Panicum maximum (guineagrass), Paspalum dilatatum (dallisgrass), Phragmites spp. (reed), Sorghum halepense (ohnsongrass) and Typha spp. (cattail). Other perennial species not listed above on which the method and compositions of the invention can be employed include, without limitation, Equisetum spp. (horsetail), Pteridium aquilinum (bracken), Rubus spp. (blackberry) and Ulex europaeus (gorse).

### EXAMPLES

The examples described below are provided to illustrate the present invention and are not included for the purpose of limiting the invention.

### Example 1

Sweet corn, bush beans, broccoli, and geranium are treated with NAC amide as a 10⁻⁵ M solution in water. NAC amide may be prepared, for example, as described in U.S. Pat. No. 6,420,429 to D. Atlas et al., the contents of which are incorporated by reference herein. Control plants treated with water alone are also included. Treatment is accomplished by a single spray of the entire above-ground plant surface so as to thoroughly wet it to the run-off point. At pre-selected times (72, 96, 120 and 168 hours) after treatment, randomly chosen leaves are selected from each plant (treatment or control); and standardized discs cut from each leaf are bioassayed to measure the degree of inducible protective response. The bioassay consists of giving one 4th-instar larva of T. ni or O. nubilalis a choice of feeding on a standardized leaf disc from the treated plant versus the control plant (i.e., H₂O-treated) in a two-choice petri-dish arena under standardized environmental conditions in 24 hours or less. (Chiang, H. S. et al., Jour. Chem. Ecol., 13:741-49 (1987)). At least 15 replicate assays per treatment or control are performed. Area eaten per disc is measured in cm² using an electronic area meter to determine that the described treatment with NAC amide produces a long lasting, protective response.

### Example 2

In another experiment, the procedures are as above in Example 1, except that the treatment involves a combination of NAC amide and enhancing agents, i.e., ascorbic acid (5×10⁻⁶M) or Vitamin E (200 IU/liter) in a water spray. NAC amide treatment provides a protective response in the treated plants. Treatment of the plants with a combination of NAC amide and another antioxidant such as Vitamin C or E, may produce a protective response having a better residual effectiveness than is observed with NAC amide alone. In Examples 1 and 2, the method of application is spraying so as to thoroughly wet to the run-off point the above-ground surface of the plant with a solution of NAC amide in water. It is to be understood that application may also be made by drenching the root system or soaking the plant seeds. The use of an aqueous solution of NAC amide does not exclude the use of other solvents. Any non-phytotoxic, non-reactant agents in which NAC amide can be diluted or dispersed suffice and are considered to be embraced by the invention. Indeed, diluents which adhere to a given plant surface, are non-reactant, membrane permeable and guard NAC amide against oxidation, tend to enhance the ability of an NAC amide-containing composition to induce a protective response. The concentration of spray need not be limited to that which is exemplified. A narrow range of spray concentration is not required, as spray effectiveness can be expected at concentrations between 10⁻⁶ and 10⁻⁴ M.

### Example 3

In another study, two treatment levels of NAC amide are applied in 1 milliliter of paraffin (white) oil in a 1-cm wide bandaid wrapped around the base of the stem of each coleus plant. The control treatment is 1 milliliter of paraffin oil in the bandaid. Assay intervals are 72, 120, 168 and 224 hours. Other aspects of the experiment were as described above. A systemic protective response may be achieved. The protective response is enhanced in magnitude and duration if NAC amide is dispersed in a non-reactant, membrane permeable carrier such as a heavy mineral oil. Although paraffin oil can be used as carrier, other types of oils such as white oil or liquid petrolatum can be employed as well. Regardless of the specific type of carrier used, caution should be taken to assure that the carrier contains no stabilizers or other materials that may react with the antioxidant properties of NAC amide.

### Example 4

Treatment of Fraxinus spp. (ash), Quercus spp. (oaks) and Gleditsia triacanthos L. (honey locust) trees, which have a trunk circumference of 10 centimeters (cm) at 35 cm above the ground surface and a height of 4 meters, with a 10×8.3-cm trunk-banding gauze bandage, which bears 0.5 ml of paraffin oil per cm² and containing NAC amide, is expected to reduce (P<0.05 or better) reduce the amount of leaf area (cm²) eaten by assay insects, Malacosoma disstria Hubner (the forest tent caterpillar) or Lymantria disoar L. (the gypsy moth larva), compared with the leaf area eaten on control (non-stressed) trees. Residual effectiveness of NAC amide is expected to be 4-14 days per treatment. A procedure for determining dosage of antioxidant that is applied to a plant via a stem bandage is disclosed in U.S. Pat. No. 6,172,004 to R. J. Brinkler et al.

### Example 5

A High Throughput Screening (HTS) protocol can be used to evaluate the properties of protecting or tolerizing plants to biotic and abiotic stress by the NAC amide antioxidant. The HTS screen in effect measures the amount of regrowth experienced by barley plants that have been treated with an herbicidal formulation and subsequently clipped back to a height one centimeter above soil level. The actual procedures of the HTS screen are described.

Three to five barley seeds are placed in a 50 mm square pot containing a growth medium of 50% Metro-Mix 350, 25% SA1 sand and 25% Bacto Mix. Additionally, Osmocote® fertilizer is applied at a rate of 3.53 kg/m³. The pots are watered by sub-irrigation for the entire test period. Shortly after emergence, the pots are hand trimmed to two plants per pot. Greenhouse conditions consisting of a day/night temperature range of 29° C./21° C. with a 12 to 14 hour photoperiod are employed.

When the barley is approximately five inches to 12-15 cm tall, which is generally eight to nine days after planting, the plants are treated with the desired NAC amide formulation. Applications are performed using a track sprayer fitted either with an 8001E flat fan nozzle operating at a pressure of 172 kPa, or with a 9501E flat fan nozzle operating at a pressure of 166 kPa. The spray volume is equivalent to 187 liters per hectare (1/ha). The treated plants are returned to the greenhouse. Forty-eight hours after treatment, all plants, including those treated with NAC amide and untreated controls are clipped to a height of one centimeter above soil level.

Six pots containing two plants each are used to evaluate the effects of each treatment. Three days after the plants have been clipped, the plants are quantitatively measured for regrowth of the barley. Regrowth of the barley is measured from the point where the barley was clipped to the tip of the new growth. Each plant is measured separately. The recorded height of the treatment is the average height of the twelve individual plants. If desired, statistical analysis is performed using analysis of variance (ANOVA) at the 95% confidence level.

Dilute aqueous NAC amide-containing compositions are used to treat the test plants. The aqueous compositions are generally prepared by dispersing a solution of NAC amide into water. Enhancing agents can be included to obtain particular yet varied rates, i.e., they are not incorporated as a set ratio to NAC amide that vary proportionately with the rate of NAC amide, but rather are varied independently of the NAC amide rate. The rate of the enhancing agent in each instance is given in g/ha. Each experiment is performed using a particular NAC amide concentration and a particular enhancing agent, when used, at a particular application rates. The tests are performed by varying the type and amount of NAC amide, with or without an enhancing agent. For example, for each test that utilized a single enhancing agent, the concentration of both NAC amide and enhancing agent is varied.

The contents of all patent applications, published applications, patents, texts, and literature references cited in this specification are hereby incorporated herein by reference in their entirety to more fully describe the state of the art to which the present invention pertains.

As various changes can be made in the above methods and compositions without departing from the scope and spirit of the invention as described, it is intended that all subject matter contained in the above description, shown in the accompanying drawings, or defined in the appended claims be interpreted as illustrative, and not in a limiting sense.

What is claimed is:

1. A method for increasing the resistance or tolerance of a plant to a biotic or abiotic environmental stress comprising the step of administering to the surface of said plant N-acetylcysteine amide (NAC amide) in an amount effective to induce said resistance or tolerance in the plant.

2. The method according to claim 1, wherein the biotic environmental stress is selected from one or more of pests or pathogens.

3. The method according to claim 2, wherein the pests comprise insects, arachnids, or nematodes.

4. The method according to claim 2, wherein the pathogens comprise bacteria, viruses, fungi, or mycoplasms.

5. The method according to claim 1, wherein the abiotic environmental stress is an extreme in temperature or weather conditions.

6. The method according to claim 5, wherein the stress comprises one or more of drought, frost, rain, hail, moisture, humidity, or heat.

7. The method according to claim 1, wherein the stress comprises excess salinity, excess minerals, poor nutrients in soil, poor nutrients in growth medium.

8. The method according to claim 1, further comprising one or more enhancing agents.

9. The method according to claim 8, wherein the one or more enhancing agents comprise anthraquinone compounds, ascorbates, tocopherols, Vitamin C or Vitamin E.

10. The method according to claim 1, wherein the surface of the plant comprises foliage, leaves, stems, roots, flowers, buds, and stalks of the plant.

11. The method according to claim 1, wherein the NAC amide is administered by drenching the root system of the plant, spraying the plant, or direct application to the surface of the plant.

12. A method for increasing the resistance or tolerance of a plant to one or more of pests, pathogens, or abiotic environmental stresses, comprising the step of spraying the above ground surface of the plant with a solution containing N-acetylcysteine amide (NAC amide) in an amount effective to increase the plant's resistance or tolerance.

13. The method according to claim 12, wherein the NAC amide is present in an amount of between 10⁻⁶ and 10⁻⁴ M.

14. The method according to claim 12, wherein the pests comprise insects, arachnids, or nematodes.

15. The method according to claim 12, wherein the pathogens comprise bacteria, viruses, fungi, or mycoplasms.

16. The method according to claim 12, wherein the abiotic environmental stress is an extreme in temperature or weather conditions.

17. The method according to claim 16, wherein the stress comprises one or more of drought, frost, rain, hail, moisture, humidity, or heat.

18. The method according to claim 16, wherein the stress comprises excess salinity, excess minerals, poor nutrients in soil, poor nutrients in growth medium.

19. The method according to claim 12, further including an enhancing agent.

20. The method according to claim 19, wherein the enhancing agent comprises one or more environmentally safe antioxidants selected from anthraquinone compounds, ascorbates, tocopherols, Vitamin C or Vitamin E.

21. A method for increasing the resistance or tolerance of a plant to one or more of pests, pathogens, or abiotic environmental stresses, comprising the steps of applying N-acetylcysteine amide (NAC amide) to the stem of a plant in an amount sufficient to increase the plant's resistance or tolerance to said one or more pest, pathogen, or abiotic environmental stress.

22. The method according to claim 21, wherein the pests comprise insects, arachnids, or nematodes.

23. The method according to claim 21, wherein the pathogens comprise bacteria, viruses, fingi, or mycoplasms.

24. The method according to claim 21, wherein the abiotic environmental stress is an extreme in temperature or weather conditions.

25. The method according to claim 24, wherein the stress comprises one or more of drought, frost, rain, hail, moisture, humidity, or heat.

26. The method according to claim 24, wherein the stress comprises excess salinity, excess minerals, poor nutrients in soil, poor nutrients in growth medium.

27. The method according to claim 21, further comprising one or more enhancing agents.

28. The method according to claim 27, wherein the one or more enhancing agents comprise anthraquinone compounds, ascorbates, tocopherols, Vitamin C or Vitamin E.

29. A process for treating a plant or crop with exogenous N-acetylcysteine amide (NAC amide) or a derivative, salt or ester thereof, alone or in combination with one or more enhancing agents, comprising the steps of (a) applying to surfaces or foliage of the plant or crop a composition comprising NAC amide, or a derivative, salt or ester thereof; and (b) applying a biologically effective amount of the NAC amide-containing composition to the same plant or crop surfaces or foliage.

30. The process according to claim 29, wherein the NAC amide is water-soluble.

31. The process according to claim 29, wherein the enhancing agent is present a substantially non-phytotoxic amount that does not substantially antagonize or depress the biological effectiveness of the NAC amide.

32. The process according to claim 29, wherein the one or more enhancing agents comprise one or more of anthraquinone compounds, ascorbates, tocopherols, Vitamin C or Vitamin E.

33. The method according to claim 29, wherein the surface of the plant or crop comprises leaves, stems, roots, flowers, buds, and stalks.

34. The method according to claim 29, wherein the NAC amide is applied by drenching the root system of the plant or crop, spraying the plant or crop, or direct application to the surface of the plant or crop.

35. A method of enhancing the ability of a plant or plant material to withstand oxidative stress by supplementing glutathione naturally produced in the plant or plant material, comprising supplying NAC amide to the plant or plant material in an amount effective to enhance the ability of the plant or plant material to withstand oxidative stress.

36. The method according to claim 34, wherein the plant or plant material is a transgenic plant or plant material.

37. The method according to claim 34, wherein the plant material is selected from a seed, a fruit, or a cutting from the plant.

38. The method according to claim 34, wherein the NAC amide replaces defective glutathione production by the plant or plant material.

39. The method according to claim 34, wherein the NAC amide supplements glutathione production by the plant or plant material.

40. The method according to claim 34, wherein the NAC amide is supplied to the plant or plant material by drenching the root system of the plant, spraying the plant or plant material, or direct application to the surface of the plant or plant material.

41. A method of augmenting the natural defense and immune systems of a plant to protect and defend the plant against environmental, physiological and oxidative stresses and insults, comprising: providing to the plant NAC amide in combination with salicylic acid-binding protein 2 (SABP2) in an amount effective to protect and defend the plant.

42. The method according to claim 41, wherein the NAC amide in combination with SABP2 is provided by drenching the root system of the plant, spraying the plant, or direct application to the surface of the plant.

43. A method of producing a plant, a plant part, plant material or product thereof, that is healthier for consumption, comprising treating the plant, the plant part, or plant material with NAC amide in an amount effective to increase or augment the content or amount of antioxidants naturally produced in the plant, the plant part, or the plant material.

44. The method according to claim 43, wherein the plant material is a seed or a fruit.

45. The method according to claim 43, wherein the plant part is a cutting from the plant.

46. The method according to claim 43, wherein the plant product is pulp.

47. The method according to claim 43, wherein the plant, the plant part, or plant material is treated with NAC amide by drenching the root system of the plant, spraying the plant, the plant part, or plant material, or direct application to the surface of the plant, the plant part, or plant material.

48. A plant treatment composition or preparation comprising N-acetylcysteine amide (NAC amide) in an amount effective to increase the resistance or tolerance of a plant to a biotic or abiotic environmental stress.

49. The composition of claim 48, wherein the biotic environmental stress is selected from one or more of pests or pathogens.

50. The composition of claim 49, wherein the pests are selected from insects, arachnids, or nematodes.

51. The composition of claim 49, wherein the pathogens are selected from bacteria, viruses, fungi, or mycoplasms.

52. The composition of claim 48, wherein the abiotic environmental stress is an extreme in temperature or weather conditions.

53. The composition according to claim 52, wherein the stress is selected from one or more of drought, frost, rain, hail, moisture, humidity, or heat.

54. The composition according to claim 48, further including one or more enhancing agents.

55. The composition according to claim 54, wherein the one or more enhancing agents comprise anthraquinone compounds, ascorbates, tocopherols, Vitamin C or Vitamin E.

56. The composition according to claim 54, wherein the enhancing agent is present in a substantially non-phytotoxic amount that does not substantially antagonize or depress the biological effectiveness of the NAC amide.

57. The composition according to claim 48, wherein the NAC amide is water-soluble.

58. The composition according to claim 48, further comprising additional ingredients.

59. The composition according to claim 58, wherein the additional ingredients comprise solvents, surfactants, dispersants, thickening agents, antifoams, dyes, antifreezes, or preservatives.

60. The composition according to claim 58, wherein the additional ingredients comprise herbicides, plant growth regulators, or nematicides.

61. The composition according to claim 48, which is a dilute ready-to-apply solution or dispersion.

62. The composition according to claim 48, which is a concentrate composition.

63. The composition according to claim 62, wherein the concentrate composition is a solid or a liquid concentrate.

64. The composition according to claim 62, wherein the concentrate composition is selected from the group consisting of an aqueous solution, an emulsifiable concentrate, a suspension concentrate, an aqueous emulsion, an oil-in-water emulsion, a water-in-oil emulsion, or a water-in-oil-in-water emulsion.
